(19) United States
(12) Patent Application Publication
(10) Pub. No.: US 2022/0257237 A1
(43) Pub. Date: Aug. 18, 2022

(54) MEDICAL SUTURE AND METHOD FOR PRODUCING SAME

(71) Applicant: SAMYANG HOLDINGS CORPORATION, Seoul (KR)

(72) Inventors: Ho Sung LEE, Daejeon (KR); Dae Hui RYU, Daejeon (KR); Hye Sung YOON, Yongin-si (KR)

(73) Assignee: SAMYANG HOLDINGS CORPORATION, Seoul (KR)

(21) Appl. No.: 17/416,755

(22) PCT Filed: Mar. 27, 2020
(86) PCT No.: PCT/KR2020/004193

§ 371 Date: Jun. 21, 2021

(30) Foreign Application Priority Data
Oct. 31, 2019 (KR) 10-2019-0138169

(51) Int. Cl. A61B 17/04; A61B 17/06; A61L 17/10

(52) U.S. Cl. CPC A61B 17/0487; A61B 17/06066; A61B 2017/00526; A61L 17/10; A61B 2017/06176; A61B 17/06166

(57) ABSTRACT

In order to provide a means for fixing an end of a medical suture to skin tissue in a short time and thereby reduce an operation time, the present invention provides: a medical suture comprising a main body, which extends in the length direction, and a stopper, which is at one end of the main body and has a larger cross section than the main body; and a method for producing same.

### TECHNICAL FIELD

The present invention relates to a medical thread and a method for manufacturing the same.

### BACKGROUND ART

In general, medical threads are used for various purposes including sealing or suturing damaged parts of tissues, such as skin, muscles, tendons, internal organs, bone tissues, nerves, blood vessels and the like, as well as for closing and binding together tissues in surgical incisions during surgical interventions, for repair, support and/or fixation of bodily tissues.

When using conventional medical threads in order to stitch incised tissues, formation of a knot is required. However, there are various knotting methods and since some of them are quite complicated, application of conventional medical threads demands preliminary training. Another problem is that knotting consumes a considerable share in the total duration of the surgical procedure. Accordingly, a need to develop a medical thread that can be used without forming a knot has emerged.

To address the need for knotless thread a barbed thread has been developed. Barbed thread can be classified into a bidirectional type having barbs formed in both directions and a unidirectional type having barbs formed in one direction only. Since the former bidirectional type has barbs facing in opposite directions, suturing starts from the center of the wound and continues in both directions to the end of the incision, bi-directional barbed threads obviate the need to secure the end of the thread with the knot. The latter unidirectional type, however, has barbs resisting movement, when in tissue, in the direction only that is opposite from the direction in which the barbs face, therefore a means for fixing the medical thread at the end of the incision is necessary.

In order to fix the end of such a unidirectional type barbed thread, the anchoring effect is achieved by processing the end part in a loop shape, stitching the tissue with suture needle and approaching the loop close to the tissue, one more stitching and passing the suture needle through the loop, and tightening the thread. Prior art is the passing a thread through a small loop molded at the end of the unidirectional type barbed thread with the consequent tightening of the loop to achieve the anchoring effect. A solution of reducing complexity and inconvenience of the procedure and reducing time required to fix the end of the unidirectional barbed thread is demanded.

### PRIOR ART PUBLICATION

### Patent Publication

Korean Patent Laid-open Publication No. 10-2019-0061944 (published on 2019 Jun. 5 )

### CONTENTS OF THE INVENTION

### Problems to be Solved

The present invention is to resolve the problem of prior art as stated above. Accordingly, the purpose of the present invention is to provide a medical thread that allows for minimizing the time of the surgical procedure by providing a means for fixing the end of the medical thread to skin tissue in a short time, and a method for manufacturing the same.

Another purpose of the present invention is to provide a medical thread that can minimize the pain of the patient without its detachment from or passing through the skin tissue.

### Technical Means

An aspect of the present invention provides a medical thread comprising: a body extended along the longitudinal direction; and at one end of the body, a stopper having a cross section larger than the cross section of the body.

In an embodiment, the stopper can be placed perpendicularly to the longitudinal direction of the body adjacent thereto, and have a shape of triangular prism or triangular pyramid.

Also, in an embodiment, the stopper can have a thickness of 0.1 mm to 5.0 mm, and a side of the stopper can have a length of 2 mm to 5 mm.

Also, in an embodiment, the body can be connected to the center of the stopper, and the edge angle of the stopper can be 60°.

Also, in an embodiment, the attachment strength between the body and the stopper can be 0.3 kgf to 10.0 kgf.

Also, in an embodiment, each of the body and the stopper independently can comprise one or more polymers selected from polydioxanone (PDO), polycaprolactone (PCL), polylactic acid (PLA), polyglycolic acid (PGA), polytrimethylcarbonate (PTMC), polypropylene (PP), Nylon and polytetrafluoroethylene (PTFE), and copolymers thereof.

Also, in an embodiment, the other end of the body can be combined with a surgical needle that can be inserted into the surgical site.

Also, in an embodiment, the surface of the body can comprise plural barbs protruding outward (or projecting from the body).

Another aspect of the present invention provides a method for manufacturing a medical thread, comprising the steps of: preparing a body extended along the longitudinal direction; heating and melting one end of the body; injecting the melted end of the body into a mold having a predetermined shape to form a stopper at the end of the body; cooling and separating the body and the stopper from the mold, wherein the stopper has a cross section larger than the cross section of the body.

In an embodiment, the method for manufacturing a medical thread can further comprise step of adjusting the position of the stopper relative to the melted end of the body.

Also, in an embodiment, in the step of cooling and separating the body and the stopper from the mold, the body and the stopper can be cooled with a cooling air amount of 0.01 to 3.0 mpa for a time of 5.0 to 120 seconds.

Also, in an embodiment, in the step of heating and melting one end of the body, the heating temperature can be 50 to 400° C.

### Effect of the Invention

According to the present invention, a means capable of fixing an end of medical thread to skin tissue in a minimized time is provided, and thereby the duration of surgical procedure can be reduced, and the effect of minimizing the pain and feeling of foreign body in the patient without its detachment from or passing through skin tissue can be achieved.

### BRIEF EXPLANATION OF THE DRAWINGS

FIG. 1 shows an embodiment of the medical thread according to the present invention.

FIG. 2 is an enlarged drawing of the stopper (20) of the medical thread shown in FIG. 1.

FIG. 3 is an enlarged drawing of a part of the body (10) of the medical thread shown in FIG. 1.

FIG. 4 is a schematic drawing of the front view of the body (10) of the medical thread shown in FIG. 1.

FIG. 5 shows a process flow of a method for manufacturing the medical thread shown in FIG. 1.

### CONCRETE MODE FOR CARRYING OUT THE INVENTION

The present invention is explained in detail below with reference to the figures for the concrete embodiments thereof. However, the present invention is not limited to such concrete embodiments. In addition, in explaining the present invention, detailed explanation about known constitutions or functions irrelevant to the gist of the present invention could be omitted, but the scope of the present invention should not be interpreted as being limited by such omission.

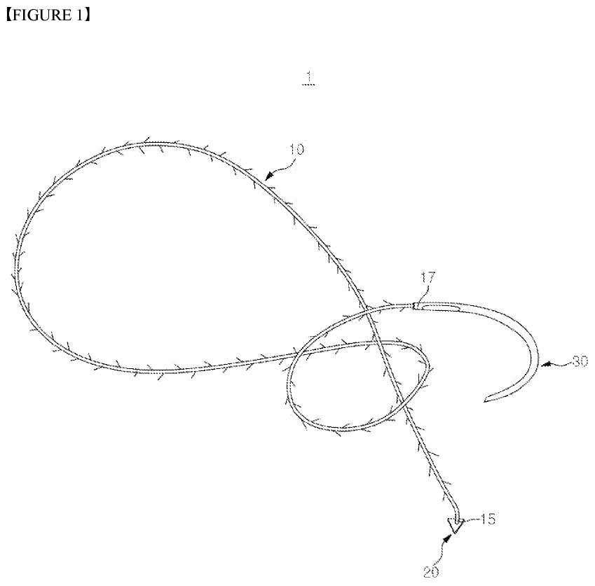

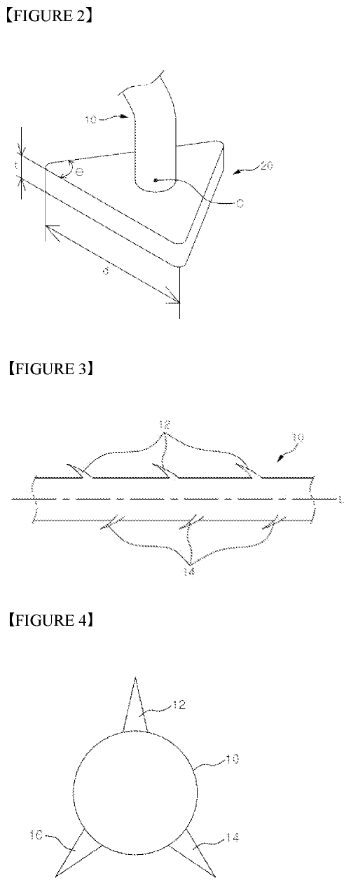

FIG. 1 shows an embodiment of the medical thread according to the present invention, FIG. 2 is an enlarged drawing of the stopper (20) of the medical thread shown in FIG. 1, FIG. 3 is an enlarged drawing of a part of the body (10) of the medical thread shown in FIG. 1, and FIG. 4 is a schematic drawing of the front view of the body (10) of the medical thread shown in FIG. 1.

With reference to FIGS. 1 to 4, an embodiment of the present invention can provide a medical thread (1) comprising: a body (10) extended along the longitudinal direction and comprising plural barbs protruding outward (or projecting from the body); and at one end of the body (10), a stopper (20) having a cross section larger than the cross section of the body (10).

A medical thread (1) according to an embodiment of the present invention can be a thread for use in suturing the part of the tissue wounded owing to surgical intervention or external injury, or a thread inserted into skin tissue and used for skin tightening.

The body (10) can be formed in a thin and long shape extended along the longitudinal direction, and can have flexible property so that it may be deformed by external force. In this regard, the longitudinal direction can be understood as a direction of extending of the center axis (L) of the body (10) in FIG. 3.

In the outer side of the body (10), plural barbs (12, 14, 16) tangentially (or obliquely) inclined in the longitudinal direction can be provided.

The plural barbs (12, 14, 16) can be formed on the surface of the body (10) with a predetermined angle to the longitudinal direction of the body (10). For example, the plural barbs (12, 14, 16) can be angulated to the longitudinal direction of the body at 10° to 45° angles. By forming the plural barbs (12, 14, 16) having a predetermined angle on the surface of the body (10) as such, the plural barbs (12, 14, 16) can be easily entangled to the skin tissue. The plural barbs (12, 14, 16) herein can be formed along one direction (unidirectional type). Thus, the medical thread (1) of the present embodiments can be a unidirectional type barbed thread.

Concretely, a first barb (12) can be formed on the surface of one side of the body (10), and a second barb (14) and a third barb (16) can be formed on the surface of the other side of the body (10), spaced in circumferential direction from the first barb (12). Also, the first barb (12), the second barb (14) and the third barb (16) can be formed in plurality, and each of them can be aligned (or arranged) in a row along the longitudinal direction of the body (10).

In the present embodiments, as shown in FIG. 4, the medical thread can be equipped with three barbs (12, 14, 16) or with two barbs or four or more barbs arranged in circumferential direction relative to the cross section of the body (10).

Also, the first barb (12) and the second barb (14) can be formed to have different angles relative to one other. Although a medical thread having only the first barb (12) and the second barb (14) formed on the surface of the body (10) is exemplified and explained in the present embodiments, it can further comprise plural barbs placed on the other sides.

Also, the pattern of the barbs (12, 14, 16) formed is not limited thereto, and the barbs can be arranged in a spiral pattern along the circumference of the body (10).

A stopper (20) having a cross section larger than the cross section of the body (10) can be provided at one end of the body (10). For example, the stopper (20) can be provided in a shape of triangular prism or triangular pyramid, and it can have a thickness (t) of 0.1 mm to 5.0 mm. If the thickness of the stopper (20) is less than 0.1 mm, stopper may pass through the skin tissue without entangling to the tissue. If the thickness of the stopper (20) is greater than 5.0 mm, such stopper can cause pain due to feeling of foreign body. Preferably, the stopper (20) can have a thickness (t) of 0.1 mm to 3.0 mm, or 0.1 mm to 1.0 mm.

Also, if the stopper (20) is provided in a shape of triangular prism, a side of it can have a length (d) of 2 mm to 5 mm. If the stopper (20) is provided in a shape of triangular pyramid, a side of the bottom surface of the triangular pyramid can have a length (d) of 2 mm to 5 mm.

Also, if the stopper (20) is provided in a shape of triangular prism, the edge angle made by two sides can be 5° to 87.5°. Preferably, the edge angle made by the two sides can be 60° (The cross section of the stopper (20) is equilateral triangle). If the stopper (20) is provided in a shape of triangular pyramid, in a triangle of cross section parallel to the bottom surface of the triangular pyramid, the edge angle made by two sides can be 5° to 87.5°. Preferably, the edge angle made by the two sides can be 60° (The cross section of the stopper (20) is equilateral triangle).

Also, the edge of the stopper (20) can be formed in a tapered shape. In this case, the pressure applied to skin tissue by the edge of the stopper (20) can be dispersed, consequently resulting in reduction of pain for the patient.

However, the shape of the stopper (20) is not limited thereto, and it can have a shape of cylinder, cone, polyprism (for example, quadrangular or more faceted polyprism), or polypyramid (for example, quadrangular or more faceted pyramid).

Also, the stopper (20) can be placed perpendicularly to the longitudinal direction of the body (10). That is, the cross section of the stopper (20) can be perpendicular to the body (10) and thus the medical thread can be easily entangled to the skin tissue.

Also, the center (C) of the stopper (20) can meet the longitudinal center of the body (10). In this case, the body (10) can be placed at the center of the stopper (20) and thus the medical thread can be easily entangled to the skin tissue.

By providing the stopper (20) as such, a procedure of making a knot at the end of the body (10) can be skipped resulting in shorter duration of the surgical intervention and in reduced risk that can be caused during the procedure of making a knot.

The above-explained barbs (12, 14, 16) formed on the body (10) in a unilateral direction can resist the one-directional movement of the medical thread (1), and in addition, the stopper (20) provided at the end of the body (10) can resist the counter-directional movement of the medical thread (1). That is, the medical thread (1) can be fixed in the tissue of the subject by the barbs (12, 14, 16) and the stopper (20).

According to an embodiment of the present invention, in the medical thread of the present invention, the attachment strength between the body and the stopper can be 0.3 kgf to 10.0 kgf, and more concretely, it can be 0.5 to 10.0 kgf, 0.7 to 10.0 kgf, 0.9 to 10.0 kgf, 1.1 to 10.0 kgf, 1.3 to 10.0 kgf, 1.5 to 10.0 kgf, 1.7 to 10.0 kgf, 2.0 to 10.0 kgf, 1.1 to 8.0 kgf, 1.3 to 8.0 kgf, 1.5 to 8.0 kgf, 1.7 to 8.0 kgf, 2.0 to 8.0 kgf, or 2.0 to 7.0 kgf.

The body (10) and the stopper (20) can be formed from the same material. For example, after the body (10) is formed, the stopper (20) is formed from the same material by melting the material of the body (10). This is explained in detail below.

Or, each of the body (10) and the stopper (20) can independently be formed from a bio-absorbable or non-absorbable polymer material. For example, each of the body (10) and the stopper (20) independently can comprise one or two polymers selected from polydioxanone (PDO), polycaprolactone (PCL), polylactic acid (PLA), polyglycolic acid (PGA), polytrimethylcarbonate (PTMC), polypropylene (PP), Nylon and polytetrafluoroethylene (PTFE), and copolymers thereof. Herein, polydioxanone (PDO), polycaprolactone (PCL), polylactic acid (PLA), polyglycolic acid (PGA) and polytrimethylcarbonate (PTMC) are bio-absorbable polymer materials, and polypropylene (PP), Nylon and polytetrafluoroethylene (PTFE) are non-absorbable polymer materials.

Also, the other end of the body (10) can be combined with (or attached to) a surgical needle (30), which can be inserted into surgical site.

Any kind of needle, which can penetrate bodily tissue, can be used as a surgical needle (30) without limitation to its type.

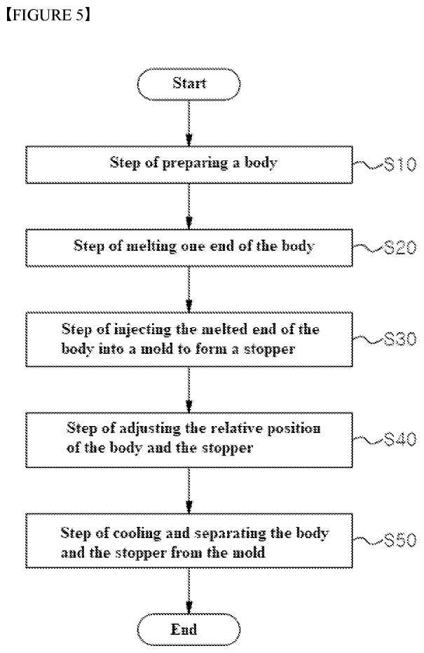

FIG. 5 represents a process flow of a method for manufacturing the medical thread (1) according to an embodiment of the present invention.

The method for manufacturing the medical thread (1) according to an embodiment of the present invention is explained below, with reference to FIG. 5.

First, the body (10) is prepared (S10), and one end of the body (10) is heated and melted (S20). Concretely, to melt the end of the body (10), the body (10) is placed in a transfer unit that can transfer the body (10), and the body (10) is injected into a pre-heated mold so that the end of the body (10) can be heated and melted.

According to an embodiment of the present invention, the step of preparing the body can further comprise a step of forming plural barbs on the surface of the body.

In an embodiment, the rate for injecting the body (10) into the mold by the transfer unit can be 0.1 to 2.0 mm/s. Herein, if the injection rate is less than 0.1 mm/s, the productivity can decline, and if it is greater than 2.0 mm/s, the injection of the body (10) might be performed faster than its melting and thereby the body (10) might bend or injection itself might fail. Preferably, the rate for injecting the body (10) into the mold by the transfer unit can be 0.1 to 1.0 mm/s.

Also, the mold can be heated by a heating unit, and the heating temperature can be 50 to 400° C. If the heating temperature is lower than 50° C., the body (10) is not melted, and if it is higher than 400° C., excessive melting of the body (10) in the mold may impede cooling, or lead to scorching and sticking of the body (10) onto the mold. Preferably, the heating temperature can be 150 to 250° C.

Thereafter, the melted end of the body (10) is injected into the mold having a predetermined shape so that the stopper (20) can be formed at the end of the body (10) (S30).

In an embodiment, the mold having a predetermined shape can be of a shape of triangular prism or triangular pyramid as explained above.

Then, after the step (S40) of adjusting the position of the stopper (20) relative to the end of the body (10), the stopper (20) can be formed so that its center (C) is positioned at the center of the body (10).

In an embodiment, the adjustment of the relative position between the stopper (20) and the body (10) can be carried out by adjusting the position of the transfer unit for the body (10) or the position of the mold.

Then, after the step (S50) of cooling and separating the body (10) and the stopper (20) from the mold, the medical thread (1) wherein the stopper (20) is formed at one end of the body (10) can be manufactured.

In an embodiment, in the cooling step, the cooling air amount can be 0.01 to 4.5 mpa, 0.01 to 4.0 mpa, 0.01 to 3.5 mpa, or 0.01 to 3.0 mpa. If the cooling air amount is less than 0.01 mpa, the cooling may be insufficient and thereby the molded product might not be discharged, and if the cooling air amount is greater than 4.5 mpa, the cooling might become excessive, which might result in reduction of attachment strength between the body (10) and the stopper (20).

More concretely, the cooling air amount can be 0.01 mpa or more, 0.02 mpa or more, 0.03 mpa or more, 0.04 mpa or more, 0.05 mpa or more, 0.06 mpa or more, 0.07 mpa or more, 0.08 mpa or more, 0.09 mpa or more or 1.0 mpa or more, and 4.5 mpa or less, 4.3 mpa or less, 4.1 mpa or less, 3.9 mpa or less, 3.7 mpa or less, 3.5 mpa or less, 3.3 mpa or less, 3.1 mpa or less or 3.0 mpa or less.

In an embodiment, in the cooling step, the cooling time can be 5.0 to 120 s, and more concretely, it can be 5.0 to 100 s, 5.0 to 80 s, 5.0 to 60 s, 5.0 to 40 s, 10.0 to 100 s, 15.0 to 80 s, 20.0 to 60 s, 25.0 to 40 s, 30.0 to 40 s, or 35 to 40 s. If the cooling time is less than 5.0 s, inappropriate shape may be formed, and if it is greater than 120 s, the production speed may decrease. According to an embodiment of the present invention, the cooling air amount can be 0.1 to 0.2 mpa, and the cooling time can be 35 to 40 s.

Meanwhile, the rate for injecting the body (10) into the mold to form the stopper (20) can vary depending on the size of the mold and the material of the body (10).

The relationship between the injection rate and the material and size of the body (10) is represented in the following Table 1.

TABLE 1

| | Diameter | | | | | | | | |
|---|---|---|---|---|---|---|---|---|---|
| | of the | | | | | | | | |
| Material | body | | | | | | | | |
| of the | (USP | Injection rate (mm/s) | | | | | | | |
| body | Size) | 0.4 | 0.5 | 0.6 | 0.7 | 0.8 | 0.9 | 1.0 | 1.1 |
| PDO | 01 | Conform | Conform | Conform | Conform | Conform | Conform | Conform | Conform |
| | 0 | Conform | Conform | Conform | Conform | Conform | Conform | Conform | Conform |
| | 20 | Conform | Conform | Conform | Conform | Conform | Conform | Conform | Conform |
| | 30 | Conform | Conform | Conform | Conform | Conform | Conform | Conform | Conform |
| | 40 | Conform | Conform | Conform | Conform | Conform | Conform | Conform | Conform |
| PGCL | 0 | Conform | Conform | Conform | Conform | Non- | Non- | Non- | Non- |
| | | | | | | Conform | Conform | Conform | Conform |
| | 20 | Conform | Conform | Conform | Conform | Non- | Non- | Non- | Non- |
| | | | | | | Conform | Conform | Conform | Conform |
| | 30 | Conform | Conform | Conform | Conform | Non- | Non- | Non- | Non- |
| | | | | | | Conform | Conform | Conform | Conform |
| | 40 | Conform | Conform | Conform | Conform | Non- | Non- | Non- | Non- |
| | | | | | | Conform | Conform | Conform | Conform |

The following Table 2 represents appearance and attachment strength of the stopper (20) depending on the cooling time during manufacturing of the stopper (20) under the fixed conditions of: 0.8 mm/s for PDO and 0.6 mm/s for PGCL with the injection rate of the body (10) varying according to the size; 0.5 mpa as the cooling air amount; and 5 s as the waiting time after completion of the injection.

TABLE 2

| | Diameter | | | |
|---|---|---|---|---|
| Material | of the body | Cooling time (mm/s) | | |
| of the body | (USP Size) | 5 | 15 | 20 |
| PDO | 1 | Non-Conform | Excellent | Excellent |
| | 0 | Non-Conform | Excellent | Excellent |
| | 20 | Non-Conform | Excellent | Excellent |
| | 30 | Non-Conform | Excellent | Excellent |
| | 40 | Non-Conform | Excellent | Excellent |

| | Diameter | | | |
|---|---|---|---|---|
| Material | of the body | Cooling time (mm/s) | | |
| of the body | (USP Size) | 30 | 35 | 40 |
| PGCL | 0 | Excellent | Excellent | Excellent |
| | 20 | Excellent | Excellent | Excellent |
| | 30 | Excellent | Excellent | Excellent |
| | 40 | Excellent | Excellent | Excellent |

The following Table 3 represents the attachment strength of the stopper manufactured with 0.8 mm/s for PDO and 0.6 mm/s for PGCL as the injection rate of the body (10) according to the body (10) size, and the cooling air amount reduced from 0.5 mpa to 0.1 mpa.

TABLE 3

| | Diameter | |
|---|---|---|
| Medical | of the body | Cooling air amount (mpa) 0.5 → 0.1 |
| thread type | (USP Size) | Stopper attachment strength |
| PDO | 01 | 58.5% increase |
| | 20 | 73.0% increase |
| | 40 | 52.6% increase |

The effect of the medical thread and method for manufacturing the same described above is explained below.

The medical thread (1) according to the present invention provides the stopper (20) at one end of the body (10), and thus a procedure of making a knot at the end of the body (10) can be skipped, which allows to minimize the duration of surgical intervention as well as reduce the risk that might be caused during the procedure of making a knot.

Also, the edge of the stopper (20) can be formed in a tapered shape. In this case, the pressure applied to skin tissue by the edge of the stopper (20) can be dispersed, and thus the pain of the patient can be reduced.

Also, the stopper (20) can be placed perpendicularly to the longitudinal direction of the body (10). That is, the cross section of the stopper (20) can be perpendicular to the body (10) and thus the medical thread can be easily entangled to the skin tissue.

Also, the center (C) of the stopper (20) can meet the longitudinal center of the body (10). In this case, the body (10) can be placed at the center of the stopper (20) and thus the medical thread (1) can provide well-balanced entanglement to the skin tissue.

The result of measuring attachment strength of the stopper to the body of of the medical thread is explained below.

1. Measurement of the attachment strength between the stopper and the body of PDO medical thread

The body of medical thread was prepared from PDO to have the diameter shown in the following Table 4, and one end thereof was heated by the method explained above under the cooling air amount condition of 0.5 mpa, to manufacture a medical thread having a stopper of triangular shape.

Next, a surgical needle was passed through a rubber for measuring Tissue Drag, and then the surgical needle was pulled until the stopper was attached to the rubber

Next, a manual tensile strength tester was set according to the measurement conditions suitable for the test sample in conformity with the the tensile strength tester manual (MTR-001-S008), after which the end of the body (10) equipped with the surgical needle was fixed in the upper grip and the rubber-attached end of the body (10) was fixed in the lower grip, and then the attachment strength was measured. The results are shown in Table 4.

As shown in Table 4, stopper attachment strength obtained from the test samples 1-1 to 1-5 did not reach the target value under the cooling air amount condition of 0.5 mpa.

Hence, Samples 1-1 to 1-5 were manufactured under the reduced cooling air amount condition of 0.1 mpa, and the attachment strength was measured by the same method as above. As a result, stopper attachment strength remarkably increased as shown in Table 4. It was demonstrated that the medical thread manufactured under the cooling air amount condition of 0.1 mpa showed improved stopper attachment strength—namely, 1.2 to 6 times greater—as compared with the medical thread manufactured under the cooling air amount condition of 0.5 mpa.

In addition, it could be seen that the PDO medical thread equipped with the stopper of the present invention has excellent attachment strength between the body and the stopper, and thus if a part of body such as skin tissue, internal organ tissue and the like is sutured by the medical thread of the present invention, excellent fixation strength can be achieved.

TABLE 4

| | | | Result | |
|---|---|---|---|---|
| | | | Stopper attachment | |
| | | | strength (kgf) | Stopper attachment |
| | | Diameter | (Cooling air | strength (kgf) |
| | USP | after barb | amount of 0.5 mpa) | (Cooling air |
| No. | size | formation | (mean/minimum) | amount of 0.1 mpa) |
| Sample | 3/0 | 4/0 | 1.10/0.38 | 2.09 |
| 1-1 | | | | |
| Sample | 2/0 | 3/0 | 2.42/1.88 | — |
| 1-2 | | | | |
| Sample | 00 | 2/0 | 2.95/2.46 | 4.04 |
| 1-3 | | | | |
| Sample | 01 | 00 | 4.05/3.40 | — |
| 1-4 | | | | |
| Sample | 02 | 01 | 4.06/3.34 | 6.94 |
| 1-5 | | | | |

2. Measurement of the attachment strength between the stopper and the body of PGCL medical thread

The body of medical thread was prepared from PGCL to have the diameter shown in the following Table 5, and one end thereof was heated by the method described above to manufacture a medical thread having a stopper of triangular shape.

Next, the attachment strength between the stopper and the body of PGCL medical thread was measured by the same measurement method as described above. The results are shown in the following Table 5.

TABLE 5

| | Diameter | Result |
|---|---|---|
| USP | after barb | Stopper attachment strength (kgf) |
| size | formation | (mean/minimum) |
| 3/0 | 4/0 | 2.822/2.264 |
| 2/0 | 3/0 | 3.455/3.133 |
| 00 | 2/0 | 4.736/4.326 |
| 01 | 00 | 5.413/4.982 |

According to Table 5, it could be confirmed that the stopper attachment strength increased as the diameter after barb formation of the PGCL medical thread increased, and the target attachment strength between the stopper and the body could be achieved sufficiently.

### Explanation of Reference Numeral

1: Medical thread

10: Body

20: Stopper

30: Surgical Needle

1. A medical thread comprising: a body extended along the longitudinal direction; and at one end of the body, a stopper having a cross section larger than the cross section of the body.

2. The medical thread of claim 1, wherein the stopper is placed perpendicularly to the longitudinal direction of the body adjacent thereto, and has a shape of triangular prism or triangular pyramid.

3. The medical thread of claim 2, wherein the stopper has a thickness of 0.1 mm to 5.0 mm, and a side of the stopper has a length of 2 mm to 5 mm.

4. The medical thread of claim 2, wherein the body is connected to the center of the stopper, and the edge angle of the stopper is 60°.

5. The medical thread of claim 1, wherein the attachment strength between the body and the stopper is 0.3 kgf to 10.0 kgf.

6. The medical thread of claim 1, wherein each of the body and the stopper independently comprises one or more polymers selected from polydioxanone (PDO), polycaprolactone (PCL), polylactic acid (PLA), polyglycolic acid (PGA), polytrimethylcarbonate (PTMC), polypropylene (PP), Nylon and polytetrafluoroethylene (PTFE), and copolymers of thereof.

7. The medical thread of claim 1, wherein the other end of the body is combined with a surgical needle that can be inserted into surgical site.

8. The medical thread of claim 1, wherein the surface of the body comprises plural barbs protruding outward.

9. A method for manufacturing a medical thread, comprising the steps of: preparing a body extended along the longitudinal direction; heating and melting one end of the body; injecting the melted end of the body into a mold having a predetermined shape to form a stopper at the end of the body; cooling and separating the body and the stopper from the mold, wherein the stopper has a cross section larger than the cross section of the body.

10. The method for manufacturing a medical thread of claim 9, further comprising a step of adjusting the position of the stopper relative to the melted end of the body.

11. The method for manufacturing a medical thread of claim 9, wherein, in the step of cooling and separating the body and the stopper from the mold, the body and the stopper is cooled with a cooling air amount of 0.01 to 3.0 mpa for a time of 5.0 to 120 seconds.

12. The method for manufacturing a medical thread of claim 9, wherein, in the step of heating and melting one end of the body, the heating temperature is 50 to 400° C.
